(19) United States
(12) Patent Application Publication
(10) Pub. No.: US 2012/0123108 A1
(43) Pub. Date: May 17, 2012

(54) Bcl Gene Primers for Detection and Fingerprinting of Bacillus anthracis

(76) Inventor: Slawomir Lukomski, Morgantown, WV (US)

(21) Appl. No.: 13/295,135

(22) Filed: Nov. 14, 2011

Related U.S. Application Data
(60) Provisional application No. 61/456,940, filed on Nov. 15, 2010.

(51) Int. Cl. C07H 21/04

(52) U.S. Cl. 536/24.33

(57) ABSTRACT

B. anthracis detection primers can be used to amplify the conserved regions of bclB alleles encoding the collagen-like proteins found in B. anthracis , as opposed to other Bacillus species, during PCR amplification of extracted Bacillus DNA or Bacillus spores. Additionally, B. anthracis strain fingerprinting primers amplify bclA-E polymorphic regions of collagen-like proteins found in B. anthracis strains. The B. anthracis strains differ in basepair size of these polymorphic coding regions of bclA-E so that strains can be discriminated based upon distinct PCR-band patterns that migrate differently in size while resolved on an agarose gel.

DRAWINGS

### CROSS-REFERENCE TO RELATED APPLICATIONS

This application claims priority from U.S. provisional application No. 61/456,940 filed on Nov. 15, 2010

### REFERENCE TO SEQUENCE LISTING, A TABLE, OR A COMPUTER PROGRAM LISTING COMPACT DISC APPENDIX

The application is filed with a computer readable form of a sequence listing. The listing is the same as described in the specification (Table 3).

### BRIEF DESCRIPTION OF THE SEVERAL VIEWS OF THE DRAWINGS

FIG. 1 is a schematic depiction of the location and orientation of the bclABCDE genes in B. anthracis strain Sterne.

FIG. 2 is a gel electrophoresis of the products of PCR amplification using DNA templates from B. anthracis strains Sterne and Ames.

FIG. 3 is a gel electrophoresis of the PCR amplification of the bcl genes from intact Bacillus spores.

FIG. 4 is a gel electrophoresis of the PCR amplification of genomic DNAs from two B. anthracis strains, Sterne and Ames, using primers flanking the bclA-E collagen-like regions Seq. ID Nos. 3-12 loaded into individual wells.

FIG. 5 is a gel electrophoresis of the PCR amplification of genomic DNAs from two B. anthracis strains, Sterne and Ames, using primers flanking the bclA-E collagen-like regions Seq. ID Nos. 3-12 loaded into single wells.

FIG. 6 is a linear model that relates the theoretical fragment length to observed amplified fragment size of B. anthracis strains Sterne and Ames.

FIG. 7 is a model of theoretical fragment length of strains Sterne and Ames where the normalized error was independent of the theoretical amplified fragment size.

FIG. 8 is an annotated dendrogram depicting the ability to distinguish among the strains using the bclA-E genes.

FIG. 9 is a gel electrophoresis of the PCR amplification of bclA-E genes of B. anthracis, B. cereus, and one B. thuringiensis and B. mycoides strains

### DETAILED DESCRIPTION OF THE INVENTION

The Bacillus cereus group organisms form a highly homogeneous subdivision of the genus Bacillus and include three main species of B. anthracis , B. cereus, and B. thuringiensis, as well as a closely related B. mycoides. Sequence polymorphisms of the collagen-like protein bcl genes allow for specific detection of B. anthracis and the use of five bcl genes present within the B. anthracis genome can allow for fingerprinting and detection of specific B. anthracis strains. The bclABCDE sequences have been studied and a unique polymorphism in the bclB alleles of the B. cereus group organisms has allowed for designing of bclB-targeting primers that allow for the specific detection of B. anthracis strains by PCR, using both genomic DNA and purified Bacillus spores. Further, by exploiting the length variation of the collagen-like (CL) regions of bcl alleles, the combined bclABCDE PCR products generate markedly different fingerprints for the B. anthracis strains, which can be utilized for B. anthracis fingerprinting.

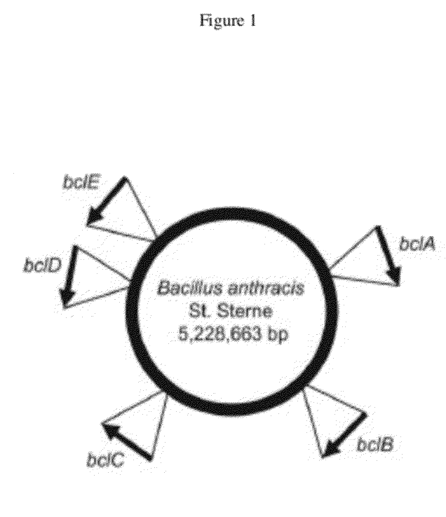

Bacillus anthracis contains five collagen-like bcl genes that are shown in FIG. 1 and Table 1. The location and orientation of the bclABCDE genes in B. anthracis strain Sterne chromosome is depicted schematically in FIG. 1. Exact nucleotide positions, locus and gene designations, and gene lengths are listed in Table 1. Table 2 presents the overall BclA-E protein architectures, as well as main characteristics, including the CL-region length variation in BclABCDE variants found among B. anthracis strains (Sterne, A1055, Vollum, USA6153, CNEVA-9066, Kruger, Ames, and Australia 94) that allows for strain fingerprinting at the gene level. Table 3 identifies the primers used for B. anthracis detection and fingerprinting.

TABLE 1

| Gene | Location | Locus | Clan | Length (bp) |
|---|---|---|---|---|
| bclA | 1,182,412-1,183,614 | BAS1130 | 7 | 1202 |
| bclB | 2,280,518-2,281,564 | BAS2281 | 2a | 1046 |
| bclC | 3,515,305-3,516,750 | BAS3557 | 4b | 1445 |
| bclD | 4,332,601-4,333,548 | BAS4423 | 6a | 947 |
| bclE | 4,518,446-4,520,389 | BAS4623 | 6a | 1943 |

TABLE 2

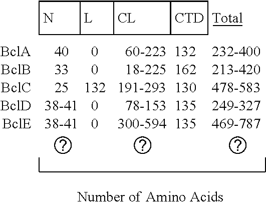

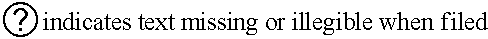

TABLE 3

| PCR target | Primer name | Sequence ID | Sequence |
|---|---|---|---|
| bclB of B. anthracis 5′ | bclB F2 | Seq. ID No 1 | AGGCCCAGAAAATATTGGAC |
| bclB of B. anthracis 3′ | bclB R4 | Seq. ID No 2 | GAGTTCCTCCCACACCTGG |
| bclA gene 5′ | bclA F1 | Seq. ID No. 3 | GAATCTTTATCAGCTAGTGCATTTG |
| bclA gene 3′ | bclA R1 | Seq. ID No. 4 | AAGCAACTTTTTCAATAATAATGGATG |
| bclB gene 5′ | bclB F1 | Seq. ID No. 5 | GGCCCAGAAAATATTGGACCTAC |
| bclB gene 3′ | bclB R1 | Seq. ID No. 6 | ATTAGACGATATTAAGACCTGCGC |
| bclC gene 5′ | bclC F1 | Seq. ID No. 7 | CCATGCTTTCCAAGTAGCGCTGG |
| bclC gene 3′ | bclC R1 | Seq. ID No. 8 | ATTAAGCGATTCTAAATACAGTTAG |
| bclD gene 5′ | bclD F1 | Seq. ID No. 9 | TGTAATAATCAAAATGGTGTACATC |
| bclD gene 3′ | bclD R1 | Seq. ID No. 10 | ATCAACTTAACCTTATTATCGTTAAC |
| bclE gene 5′ | bclE F1 | Seq. ID No. 11 | AGTCCATTAAATTCTAATTTCAAGAT |
| bclE gene 3′ | bclE R1 | Seq. ID No. 12 | ATTAACTCAATCTAATAATCGTTAAAG |

Each of the bcl genes potentially encodes a protein with an N region composed of 25 to 41 amino acids. The lengths of the collagen-like (CL) regions of each Bcl protein vary among B. anthracis strains. BclA-E proteins also contain the C-terminal domains (CTD) composed of 132-162 amino acids. In addition, only the BclC proteins contain a linker (L) region between the N and CL regions (Table 2). The genomes of B. anthracis strains contain five distinct bcl open reading frames encoding collagen-like proteins. While homologous bclA-E genes are also found in the genomes of other B. cereus group organisms, the genomes of B. anthracis strains contain a unique and differing bclB allele, which allows for specific detection of the B. anthracis. The 5′- and 3′-bclB primers identified as Seq. ID Nos. 1 and 2 (Table 3) can be used to specifically detect the DNA of B. anthracis.

Example of PCR Detection

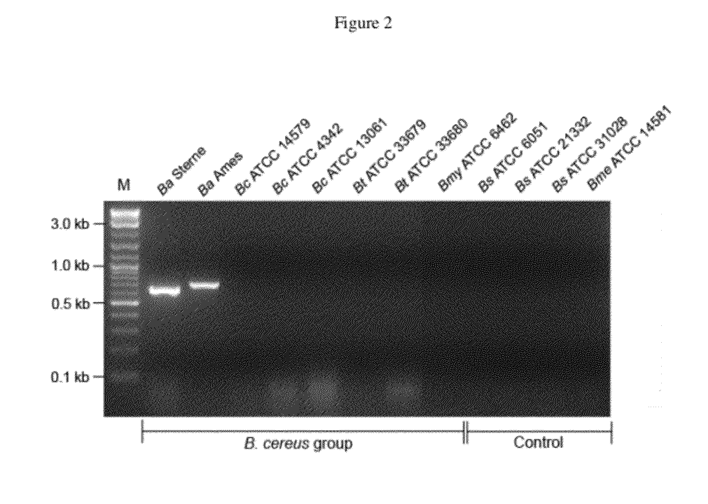

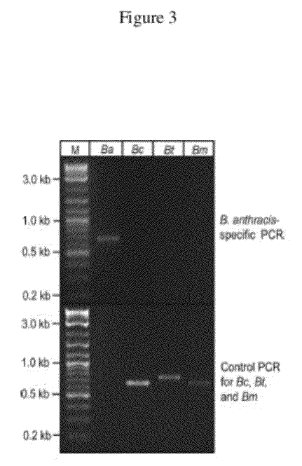

PCR amplification using DNA templates from B. anthracis (Ba) strains Sterne and Ames yielded single products of expected sizes as shown in FIG. 2. Conversely, none of the PCRs that used as templates DNA from closely-related species of three B. cereus (Bc), two B. thuringiensis (Bt), and one B. mycoides (Bmy) strains resulted in bclB-product amplification. PCR was also negative for DNA templates from the control distant strains of B. subtilis (Bs, n=3) and B. megaterium (Bme, n=1) that do not harbor the bclB gene. To assess the feasibility of a bclB-based method of detecting B. anthracis directly in the field, PCR amplification of the bcl genes from intact Bacillus spores was tested with the results shown in FIG. 3. Purified B. anthracis Sterne spores, as well as control spores from B. cereus, B. thuringiensis, and B. mycoides, were obtained and adjusted for concentration. Equal amounts of spores from each Bacillus species were added to PCR mixtures and amplification was carried out with primers that were either specific for bclB allele of B. anthracis (Seq. ID No. 1 and 2) or with control primers specific for bclB allele of other Bacillus cereus group organisms. B. anthracis spores yielded DNA products with B. anthracis -specific primers, while neither B. cereus, nor B. thuringiensis and B. mycoides spores yielded PCR products as shown in the upper panel of FIG. 3. Importantly, all of the latter spores amplified DNAs with the control specific primers, whereas B. anthracis spores did not. These data demonstrate that amplification of the bclB gene can specifically differentiate B. anthracis spores by direct PCR from spores of other B. cereus group members.

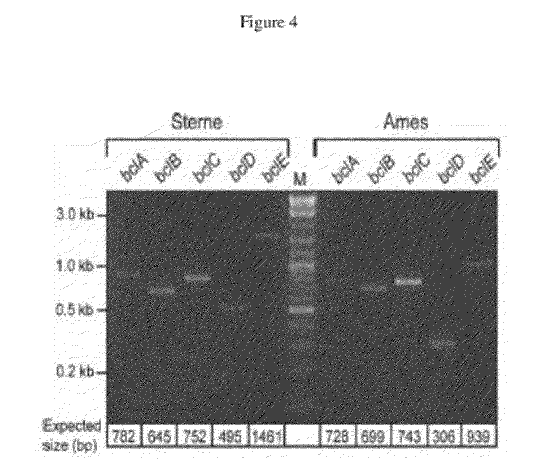

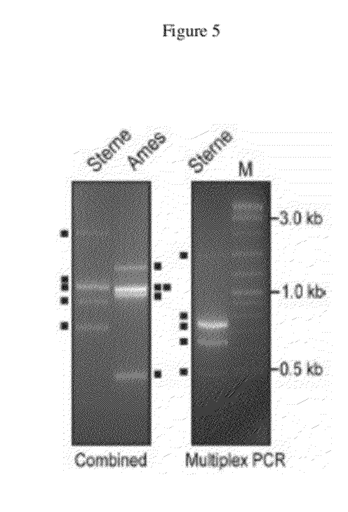

Example 2 the fingerprinting of B. anthracis strains based on bclA-E-length polymorphism. The main sequence-length polymorphism was observed within the collagen-like regions of BclA-E proteins present in various B. anthracis strains. A simultaneous analysis of the collagen- like region lengths of all five bclA-E genes can be utilized to fingerprint the different B. anthracis strains. PCRs were performed with genomic DNAs from two B. anthracis strains, Sterne and Ames, using primers flanking the bclA-E collagen-like regions which are Seq. ID Nos. 3-12. The products were individually separated in 2% agarose gels and yielded single DNA bands of the predicted sizes with the results shown in FIG. 4. The combined bclA-E-gene products, obtained from each strain, were loaded into single wells and band patterns were resolved by agarose gel electrophoresis as shown in FIG. 5 (left panel). The results in FIGS. 4 and 5 show that significantly different fingerprints are generated from the B. anthracis strain Sterne and strain Ames. In both strains, bclB-, bclD-, and bclE-amplified fragments migrated as distinct bands that significantly differed in size in each strain. The bclA- and bclC-amplification products were of ˜0.8-kb in both strains (782 by and 752 by in Sterne; 728 by and 743 by in Ames) and therefore, were not well resolved by this method. Our data demonstrate that significant length variation in the collagen-like regions of the bclA-E genes that are all present in the genomes of all available B. anthracis strains can be a powerful tool in strain fingerprinting. Finally, multiplex PCR with all five primer pairs was attempted with DNA of the Sterne strain as the template by using a temperature gradient from 50 to 65° C. for primer annealing and an Mg⁺² concentration range of 1.5 to 6.5 mM in the PCR buffer. The bcl ABCDE genes were all amplified with an annealing temperature of 50° C. and an Mg⁺² concentration of 1.8 mM, although the intensities of the bclA and bclE bands were relatively low (FIG. 5, right panel). Together, these data demonstrate that significant length variation in the CL regions of the bcl ABCDE genes that are present in the genomes of all available B. anthracis strains can be a valuable tool in strain fingerprinting.

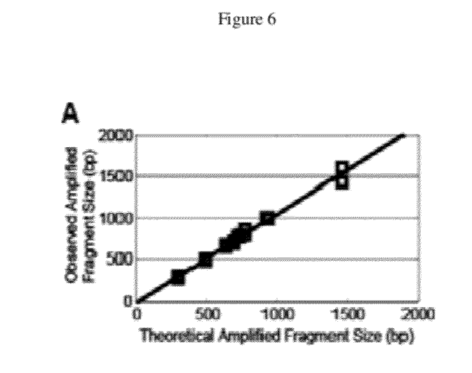

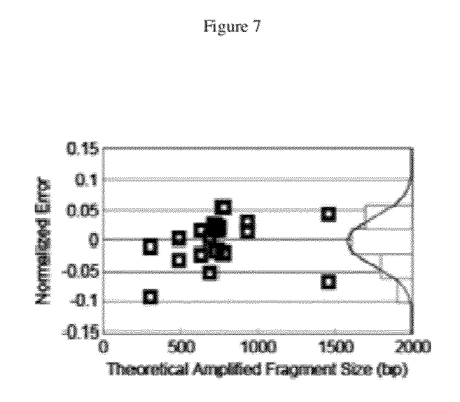

Mathematical modeling of bclA-E-based fingerprinting of B. anthracis strains. A computational approach was used to establish the feasibility of discriminating among B. anthracis strains using multiplex measurement of the sequence polymorphisms within the bclA-E genes using PCR. The first step was to develop and to calibrate a quantitative relationship between experimentally measured amplified bclA-E gene products and theoretical amplified fragment length predicted from the nucleotide sequence. The profile of amplified bclA-E gene products from the Ames and Sterne strains of B. anthracis were measured in duplicate by PCR as shown in FIGS. 4 and 5. The combined results were used to calibrate a linear model that relates the theoretical fragment length to observed amplified fragment size as shown in FIG. 6-8. The slope and intercept of the linear model were determined to be 1.059 and −18.56. In FIG. 7, the normalized error (i.e., residuals) was independent of the theoretical amplified fragment size. The distribution in the residuals, marginalized across the theoretical amplified fragment size, exhibited a normal distribution with a standard distribution(s) of 0.0374 (compare the histogram against the solid line shown on the right vertical axis of FIG. 7. The calibrated model was used to predict the fragment sizes amplified by PCR for each of the bclA-E genes observed in the genomes of six additional B. anthracis strains. The uncertainty associated with strain fingerprinting using multiplex measurement of the amplified fragments derived from the bclA-E genes was estimated using Bootstrap resampling. Bootstrap resampling was used to create a population of synthetic replicates. Each synthetic replicate was generated using the following equation:

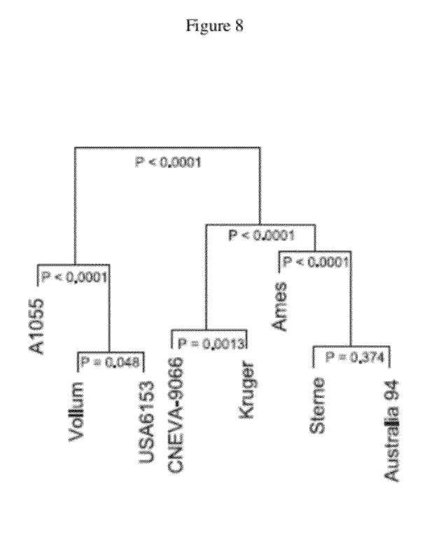

Y_{syn}(Sᵢ)=θ₁·Ŷ_{T} (Sᵢ)+θ₂+Ŷ_{T} (Sᵢ)·N(0, 0.0374),

where N(0, 0.0374) represents a random number generated from a normal distribution with a mean of zero and standard deviation of 0.0374, Si represents the theoretical amplified fragment size for each gene, and θᵢ represents the model parameters. The ability to distinguish among B. anthracis strains Sterne, A1055, Vollum, USA6153, CNEVA-9066, Kruger, Ames, and Australia 94 using the bclA-E genes was tested using this mathematical model and the levels of confidence associated with distinguishing among these strains is shown as an annotated dendrogram in FIG. 8. Hence, we predict that under the experimental conditions used here, we would be able to differentiate with confidence between two strains of B. anthracis, with the exception of the Sterne and Australia 94 strains, using a multi-locus typing approach based upon bclA-E length polymorphism.

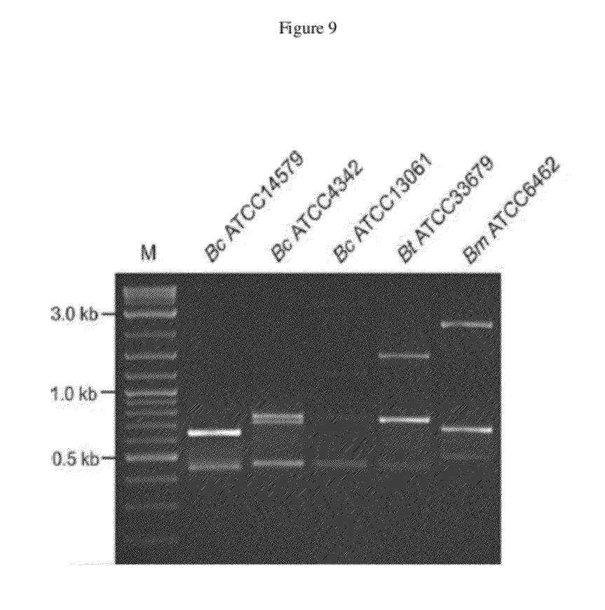

bcl gene-based fingerprinting of the B. cereus group organisms. Determination of the origin of certain spores may also be important for non-anthracis Bacillus sp in events of a hoax, blunder by the perpetrator, or psychological terrorism. Primer pairs (Seq. IDs Nos. 3-12) that were optimized for the bclA-E genes of B. anthracis were used to generate fingerprints using DNA templates from three B. cereus, and one B. thuringiensis and B. mycoides strains as shown in FIG. 9 and Table 4.

TABLE 4
Size of amplified DNA fragments (kb)

| | | | | | | Total |
|---|---|---|---|---|---|---|
| | bclA | bclB | bclC | bclD | bclE | products |
| Bc ATTC14579 | 0.48 | 0.65 | ND | 0.45 | ND | 3 |
| Bc ATTC4342 | 0.48 | 0.70 | ND | 0.78 | ND | 3 |
| Bc ATTC13061 | 0.48 | ND | 1.25 | 0.78 | 3.50 | 4 |
| Bt ATTC33879 | 0.48 | 0.70 | 1.50 | ND | ND | 3 |
| Bm ATTC6462 | ND | 0.65 | ND | 0.50 | 2.50 | 3 |

ND—Not detected

Not all primer pairs yielded bclA-E-gene products for all DNA templates, however despite partial amplification of 3-4 bands the combined PCR samples generated unique fingerprint patterns for all strains analyzed. This test demonstrates that bcl-based fingerprinting could also be employed in forensic applications for strain differentiation of all members of the Bacillus cereus group organisms.

These terms and specifications, including the examples, serve to describe the invention by example and not to limit the invention. It is expected that others will perceive differences, which, while differing from the forgoing, do not depart from the scope of the invention herein described and claimed. In particular, any of the function elements described herein may be replaced by any other known element having an equivalent function. The examples are illustrative only and show methods to produce the compounds, but are not meant to limit the production to those methods only as one skilled in the art could change the examples to produce the compounds without undue experimentation.

What is claimed:

1. An apparatus comprising B. anthracis detection primers consisting of Seq ID Nos. 1 and 2 wherein the primers amplify the conserved regions of bclB alleles encoding collagen-like proteins found in B. anthracis, as opposed to other Bacillus species, during PCR amplification of a sample wherein the sample can be either from extracted Bacillus DNA or an intact Bacillus spore.

2. An apparatus comprising B. anthracis strain fingerprinting primers consisting of Seq ID Nos. 3-12 wherein the primers amplify polymorphic collagen-like regions of bclA-E genes found in B. anthracis strains wherein the strains differ in basepair size of these coding regions of bclA-E during the PCR amplification wherein B. anthracis strains can be discriminated based upon distinct band patterns of the PCR products migrating differently in size while resolved by agarose gel electrophoresis.


SEQUENCE LISTING (12 sequences)

SEQ ID NO: 1 - DNA, 20 bp, Bacillus anthracis
aggcccagaaaatattggac

SEQ ID NO: 2 - DNA, 19 bp, Bacillus anthracis
gagttcctcccacacctgg

SEQ ID NO: 3 - DNA, 25 bp, Bacillus anthracis
gaatctttatcagctagtgcatttg

SEQ ID NO: 4 - DNA, 26 bp, Bacillus anthracis
aagcaacttttcaataataatggatg

SEQ ID NO: 5 - DNA, 23 bp, Bacillus anthracis
ggcccagaaaatattggacctac

SEQ ID NO: 6 - DNA, 24 bp, Bacillus anthracis
attagacgatattaagacctgcgc

SEQ ID NO: 7 - DNA, 22 bp, Bacillus anthracis
ccatgcttccaagtagcgctgg

SEQ ID NO: 8 - DNA, 25 bp, Bacillus anthracis
attaagcgattctaaatacagttag

SEQ ID NO: 9 - DNA, 25 bp, Bacillus anthracis
tgtaataatcaaaatggtgtacatc

SEQ ID NO: 10 - DNA, 26 bp, Bacillus anthracis
atcaacttaaccttattatcgttaac

SEQ ID NO: 11 - DNA, 26 bp, Bacillus anthracis
agtccattaaattctaatttcaagat

SEQ ID NO: 12 - DNA, 27 bp, Bacillus anthracis
attaactcaatctaataatcgttaaag